(19) United States
(12) Patent Application Publication
(10) Pub. No.: US 2006/0074315 A1
(43) Pub. Date: Apr. 6, 2006

(54) Medical diagnostic ultrasound characterization of cardiac motion

(76) Inventors: Jianming Liang, Paoli, PA (US); Sriram Krishnan, Exton, PA (US); R. Bharat Rao, Berwyn, PA (US)

Correspondence Address: SIEMENS CORPORATION;INTELLECTUAL PROPERTY DEPARTMENT, 170 WOOD AVENUE SOUTH, ISELIN, NJ, 08830, US

(21) Appl. No.: 11/184,598

(22) Filed: Jul. 19, 2005

Related U.S. Application Data
(60) Provisional application No. 60/615,616, filed on Oct. 4, 2004.

(51) Int. Cl. A61B 8/02

(52) U.S. Cl. 600/450

(57) ABSTRACT

Motion is automatically characterized from ultrasound information. Ultrasound information associated with particular time periods relative to a cycle, such as the cardiac cycle, are extracted, such as identifying and extracting ultrasound information associated with systole. By tracking an area of the heart, such as an area within the endocardial contour, the heart cycle time periods are identified. Spatial parameter values are determined as a function of time from the extracted ultrasound information. For example, the timing of motion, the eigen motion, the curvature, the local ejection-fraction ratio and/or the bending energy of parts of the cardiac tissue are determined. The spatial parameter values characterize the motion.

### RELATED APPLICATIONS

The present patent document claims the benefit of the filing date under 35 U.S.C. §119(e) of Provisional U.S. Patent Application Ser. No. 60/615,616, filed Oct. 4, 2004, which is hereby incorporated by reference.

### BACKGROUND

This present invention relates to characterizing cardiac motion from ultrasound information. Cardiac motion abnormalities may assist with diagnosis. Ultrasound imaging provides a sequence of images of the heart. The changes in tissue location from image to image show motion. The sequence of images is analyzed by a viewer to assist with diagnosis. A number of features are used to characterize the cardiac motion in order to detect cardiac motion abnormalities. For example, ejection-fraction ratio, radial displacement, velocity, thickness and thickening.

### BRIEF SUMMARY

By way of introduction, the preferred embodiments described below include methods, computer readable media and systems for automatic characterizing motion, such as cardiac motion, from ultrasound information. Ultrasound information associated with particular time periods relative to the motion cycle are extracted, such as identifying and extracting ultrasound information associated with systole in cardiac imaging using the ultrasound information. By tracking an area of the heart or other organ, such as an area within the endocardial contour, the cycle time periods are identified.

Spatial parameter values are determined as a function of time from the extracted ultrasound information. For example, the timing of motion, the eigen motion, the curvature, the local ejection-fraction ratio and/or the bending energy of parts of the cardiac tissue are determined. The spatial parameter values characterize the cardiac or other motion.

In a first aspect, a method is provided for identifying motion information from ultrasound information. Cavity area is calculated as a function of time from ultrasound frames of data. A cycle parameter is identified as a function of a change in the cavity area.

In a second aspect, a method is provided for characterizing motion from ultrasound information. A first point associated with tissue is tracked in a sequence of ultrasound data representing at least a portion of a heart or other organ. A spatial parameter value is determined for the first point as a function of time based on the tracking. Motion is characterized as a function of the spatial parameter value.

In a third aspect, a computer readable storage media has stored therein data representing instructions executable by a programmed processor for characterizing cardiac motion from ultrasound information. The instructions are for: tracking a first point associated with cardiac tissue in a sequence of ultrasound data representing at least a portion of a heart; determining a spatial parameter value for the first point as a function of time based on the tracking; and characterizing cardiac motion as a function of the spatial parameter value.

In a fourth aspect, a method is provided for characterizing motion from ultrasound information. A first point associated with tissue is tracked in a sequence of ultrasound data representing at least a portion of a heart or other organ. Two or more different types of parameter values are determined for the first point as a function of time based on the tracking. Motion is characterized as a function of the two or more different types of parameter values.

The present invention is defined by the following claims, and nothing in this section should be taken as a limitation on those claims. Further aspects and advantages of the invention are discussed below in conjunction with the preferred embodiments.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is a block diagram of one embodiment of a system for characterizing cardiac motion with an ultrasound information;

FIG. 2 is a flow chart diagram of one embodiment of a method for identifying heart cycle time periods from cardiac area;

FIG. 3A is a graphical representation of one embodiment of area as a function of time over more than one cardiac cycle, and FIG. 3B is a graphical representation of a systole time period identified from FIG. 3A;

FIG. 4 is a flow chart diagram of one embodiment of a method for characterizing cardiac motion with ultrasound data;

FIG. 5A is a graphical representation of motion tracked for a plurality of points during systole, and FIG. 5B is a graphical representation of variation in distance as a function of time of the points of FIG. 5A;

FIG. 6 is a graphical representation of one embodiment of spatial relationships used for a local ejection-fraction ratio; and

FIG. 7 is a graphical representation of one embodiment of a piece-wise sinusoidal function representing a cardiac cycle.

### DETAILED DESCRIPTION OF THE DRAWINGS AND PRESENTLY PREFERRED EMBODIMENTS

Motion abnormalities, such as cardiac motion abnormalities, may be identified by spatial parameter values, including timing, eigen motion, curvature, local ejection-fraction ratio, and/or bending energy. Each of the spatial parameter values is associated with different aspects of motion. The spatial parameter values are determined from ultrasound data, including 2D data (2D +time) or 3D data (3D +time). In addition, data from other imaging modalities may be used, such as magnetic resonance, computed tomography, x-ray, flouro x-ray, positron emission, or other now known or later developed medical imaging modes.

FIG. 1 shows a system 10 for characterizing cardiac motion. The system, methods and instructions herein may instead or additionally be used for other cyclical or repetitive motion characterization, such as analysis of diaphragm motion or a gait while jogging. In yet other embodiments, non-medical analysis is performed using the methods, systems, or instructions disclosed herein, such as analysis of turbine blade vibrations or structural reaction to environmental conditions (e.g., bridge variation due to wind). The medical imaging cardiac example is used herein.

The system 10 includes a processor 12, a memory 14 and a display 16. Additional, different or fewer components may be provided. In one embodiment, the system 10 is a medical diagnostic imaging system, such as an ultrasound imaging system. As or after images representing a patient's heart are acquired, the system 10 automatically characterizes the cardiac motion of the heart. In other embodiments, the system 10 is a computer, workstation or server. For example, a local or remote workstation receives images and characterizes cardiac motion.

The processor 12 is one or more general processors, digital signal processors, application specific integrated circuits, field programmable gate arrays, servers, networks, digital circuits, analog circuits, combinations thereof, or other now known or later developed device for processing medical image data. The processor 12 implements a software program, such as code generated manually or programmed or a trained classification system. For example, the processor 12 is a classifier implementing a graphical model (e.g., Bayesian network, factor graphs, or hidden Markov models), a boosting base model, a decision tree, a neural network, combinations thereof or other now known or later developed algorithm or training classifier. The classifier is configured or trained for distinguishing between the desired groups of states or to identify options and associated probabilities.

The processor 12 is operable to calculate cardiac related information, such as calculating area, tracking points, lines or areas, identifying cardiac cycle time periods, determining spatial parameter values as a function of time, and/or characterize cardiac motion. In one embodiment, the processor 12 implements a model or trained classification system (i.e., the processor is a classifier) programmed with desired thresholds, filters or other indicators of class. For example, the processor 12 or another processor tracks one or more points and calculates spatial parameter values for each point in a first level of a hierarchal model. The processor 12 then characterizes the cardiac motion as a classifier with the spatial parameter values being used for inputs in a second level of the hierarchal model. As another example, the processor 12 is implemented using machine learning techniques, such as training a neural network using sets of training data obtained from a database of patient cases with known diagnosis. The processor 12 learns to analyze patient data and output a diagnosis. The learning may be an ongoing process or be used to program a filter or other structure implemented by the processor 12 for later existing cases. Any now known or later developed classification schemes may be used, such as cluster analysis, data association, density modeling, probability based model, a graphical model, a boosting base model, a decision tree, a neural network or combinations thereof. For example, the characterization processes, systems or instructions used in U.S. Pat. No. ______ (Publication No. 2005-0059876), the disclosure of which is incorporated herein by reference, is used. One method is described which characterizes the motion of each segment of the heart on a scale of 1-5, as per guidelines from the American Society of Echocardiography. The classification may be performed using the motion information described above.

The classifier includes a knowledge base indicating a relationship between the spatial parameter values and/or other information. The knowledge base is learned, such as parameters from machine training, or programmed based on studies or research. The knowledge base may be disease, institution, or user specific, such as including procedures or guidelines implemented by a hospital. The knowledge base may be parameters or software defining a learned model.

The memory 14 is a computer readable storage media. Computer readable storage media include various types of volatile and non-volatile storage media, including but not limited to random access memory, read-only memory, programmable read-only memory, electrically programmable read-only memory, electrically erasable read-only memory, flash memory, magnetic tape or disk, optical media and the like. The memory 14 stores the ultrasound or image data for or during processing by the processor 12. For example, ultrasound data is a sequence of B-mode images representing a myocardium at different times. The sequences are in a clip stored in a CINE loop, DICOM images or other format. The ultrasound data is input to the processor 12 or the memory 14.

A computer readable storage medium has stored therein data representing instructions executable by a programmed processor, such as the processor 12, for automated analysis of heart function with ultrasound. The automatic or semiautomatic operations discussed herein are implemented, at least in part, by the instructions. In one embodiment, the instructions are stored on a removable media drive for reading by a medical diagnostic imaging system or a workstation networked with imaging systems. An imaging system or work station uploads the instructions. In another embodiment, the instructions are stored in a remote location for transfer through a computer network or over telephone communications to the imaging system or workstation. In yet other embodiments, the instructions are stored within the imaging system on a hard drive, random access memory, cache memory, buffer, removable media or other device.

The memory 14 is operable to store instructions executable by the programmed processor 12. The instructions are for automated analysis of heart function with ultrasound. The functions, acts or tasks illustrated in the figures or described herein are performed by the programmed processor 12 executing the instructions stored in the memory 14 or a different memory. The functions, acts or tasks are independent of the particular type of instructions set, storage media, processor or processing strategy and may be performed by software, hardware, integrated circuits, film-ware, micro-code and the like, operating alone or in combination. Likewise, processing strategies may include multiprocessing, multitasking, parallel processing and the like.

In one embodiment, the memory 14 is a computer readable storage media having stored therein data representing instructions executable by the processor 12 for characterizing cardiac motion from ultrasound information. The instructions are for tracking a first point associated with cardiac tissue in a sequence of ultrasound images or data representing at least a portion of a heart. The processor 12 determines a spatial parameter value for the first point as a function of time based on the tracking in response to further instructions. Yet other instructions cause the processor 12 to characterize cardiac motion as a function of the spatial parameter value, such as classifying the cardiac motion as a function of the spatial parameter value.

The instructions are for any or some of the functions or acts described herein. For example, in response to the instructions, the processor 12 calculates timing information automatically. A distance from a centroid to a tracked point is determined as a function of time, and a synchronicity of variation of the distance is determined as a function of time with a cardiac cycle. Alternatively, a number of tracked tissue locations within, outside or both within and outside a boundary of the cardiac tissue from a different time are determined. Out-of-place locations relative to the cardiac cycle time period may indicate abnormal motion. As another example, abnormal directions of motion are calculated automatically. Eigen values representing a direction of movement of a tracked location are calculated. Movement more equal than unequal along perpendicular directions is more likely abnormal. As yet another example, unusual variation in local curvature over time may indicate deceased cardiac tissue. A minimum, a maximum, a median, an average, a standard deviation or combinations thereof of the curvature over time may be analyzed. As another example, a local ejection-fraction is calculated. Two different local areas, such as associated with one or two segments and a centroid, are calculated as a function of tracked points on the boundary at end diastole and end systole. The local ejection-fraction ratio is a ratio of the first and second local areas. As yet another example, a bending energy of the boundary over time may indicate abnormal operation. As another example, combinations of these or other different types of parameter values are used.

In order to calculate the above or other spatial parameter values as a function of time, the image data associated with particular time periods is identified. For example, ECG information is used to identify data associated with one or more portions of or whole heart cycles. As another example, Doppler acceleration, velocity or power data is analyzed to identifying the heart cycle timing and associated data.

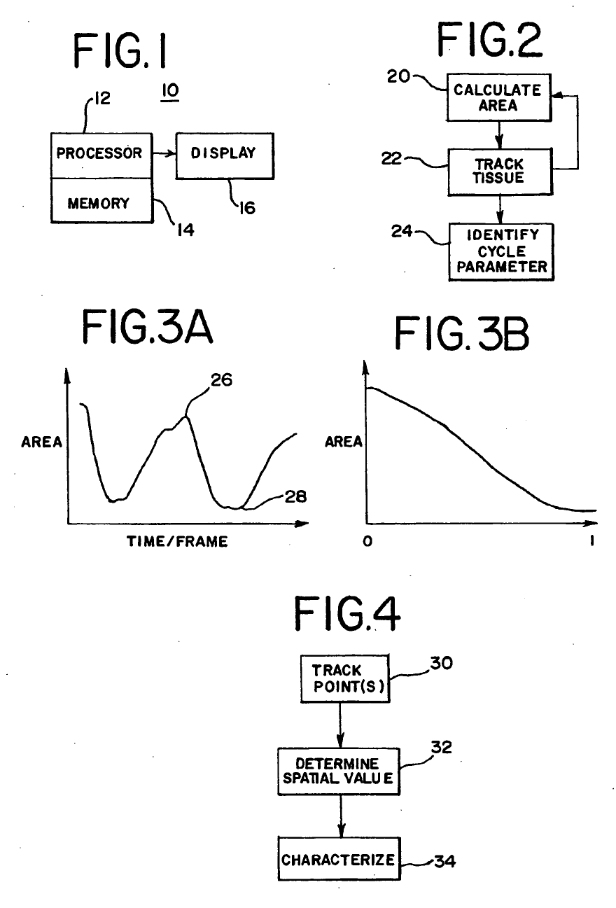

In another embodiment for use with cardiac imaging, the area or volume of the heart as a function of time is used to identify the heart cycle timing relative to the imaging data. FIG. 2 shows a method for identifying cardiac motion information from ultrasound information. Additional, different or fewer acts than shown in FIG. 2 may be used.

In, act 20, cavity area or volume is calculated as a function of time from image frames of data. “Frames of data” and “images” include data scan converted for a display with or without actual displaying of the images and/or data prior to scan conversion, such as in an acquisition polar coordinate format. The endocardial, and/or epicardial contour or tissue boundary is identified manually, automatically or semi-automatically. For example, the user identifies points along the boundary and a curve or lines between the points are determined with or without references to the image data. As another example, a filter and/or thresholds are used to automatically identify the boundary.

The tissue boundary may have one or more gaps depending on the viewing direction (e.g., A4C, A2C, or longitudinal). The gaps are closed as part of the curve fitting or line segment formation to identify the boundary. Alternatively, the gaps are identified and the tissue boundary is closed by connecting a straight or curved line between the tissue boundary points closest to the gap.

The area enclosed by the boundary is the cavity area. Using the scanning location parameters or normalized information, the actual or a representative area is calculated. For example, the cavity area of the endocardial contour is estimated. For three dimensional imaging, the cavity volume may be calculated.

The cavity area as a function of time is calculated. In act 22, the tissue associated with the boundary is tracked. In one embodiment, the procedure for identifying the tissue boundary used in act 20 is repeated for each subsequent image. Alternatively, at least a portion of a cavity border is tracked in subsequent frames of data associated with different portions of the cardiac cycle. The points along the boundary identified by the user in act 20, equally spaced points, points associated with particular tissue structures, lines and/or other locations are tracked through the sequence.

In one embodiment, the tracking disclosed in U.S. Pat. No. ______ (Publication No. 2004-0208341), filed Mar. 7, 2004, is used, the disclosure of which is incorporated herein by reference. The tracking described in this disclosure has been found to be particularly robust for tracking tissue, and extracting features such as cavit area. The tracking is performed by image analysis. For example, speckle or tissue is tracked using correlation or minimum sum of differences calculations. The best match of data for or surrounding each location is identified in subsequent images. As another example, a snake-based tracker is used. The endocardial contour for the inner border of the left ventricle wall and/or the epicardial contour for the outer border of the left ventricle wall are identified. The boundary is tracked between images based on minimum stress or distortion of the previous boundary. The relationship between the two boundaries may be used to assist in the snake-based tracker. Other now known or later developed tracking methods may be used.

For each image in the sequence, the area is calculated in act 20. Where additional images are provided in the sequence, the tissue boundary is tracked in act 22 in the additional images, and the cavity area is calculated in act 22.

FIG. 3A shows the cavity area as a function of time or frame number. The cavity area varies as a function of the cardiac cycle. A sequence of images may be associated with a portion of the cardiac cycle. For example, some examination protocols provide for images only during the systole portion of the cardiac cycles. The sequence, such as shown in FIG. 3A, may be associated with one or more cycles. For uniformity of analysis, a common portion, such as the systole or diastole portion, is extracted. The same algorithms and classifiers are used for different sequences, so extracting information associated with a common sequence or time period may more likely result in classification of input data. Alternatively, one or more cycles are identified.

In act 24, a cardiac cycle parameter is identified as a function of a change in the cavity area. For example, the ending and beginning of the systole time period are identified. End diastole and end systole correspond to maximum and minimum cavity area or volume, respectively. Inflexion points 26, 28 of the cavity area are detected as a function of time. The cavity area curve may be low pass filtered to remove any maximum or minimum associated with noise. Other processes, such as limitations on closeness in time of the inflexion points 26, 28, may be used.

Once the cardiac cycle parameter, such as end diastole, end systole, systole, diastole, r-wave, or other parameter, is identified, frames of data associated with a desired time or time period are extracted. For example, frames of data associated with systole are extracted. Decreasing cavity area between inflexion points 26, 28 represent systole, so frames of data associated with systole are identified.

For uniformity of analysis even given variation in the length of the extracted time period, the extracted frames of data are normalized as a function of time. FIG. 3B shows frames of data during a systole time period normalized. The extracted systole frames of data are re-plotted with the systole time period bounded by 0 to 1. Similarly, frames of data associated with each cardiac cycle may be normalized to a common cardiac cycle by re-plotting as function of time.

The normalized or extracted image data is used to calculate one or more feature values. The feature values indicate abnormal, normal or other characteristic of tissue motion individually or when considered as a set of two or more features. Cardiac motion may be classified as a function of the feature values. For example, tissue motion timing, eigen motion, curvature, local ejection-fraction ratio and/or bending energy are used to identify normal, abnormal or a type of abnormal operation.

FIG. 4 shows one embodiment of a method for characterizing cardiac motion from ultrasound or other imaging information. Additional, different or fewer acts may be provided.

In act 30, one or more points (single locations, lines, areas or volumes) associated with cardiac tissue is tracked in a sequence of ultrasound data representing at least a portion of a heart. For example, the tracking discussed above for act 22 of FIG. 2 is used. Different tracking may alternatively be used.

The points are tracked throughout a provided or extracted sequence, such as throughout a systole sequence, a full cardiac cycle, or a plurality of cardiac cycles. The spatial parameter values determined as a function of time from the tracked points, such as timing, eigen motion, curvature and bending energy may be calculated from systole, diastole, a full cardiac cycle or multiple cardiac cycles. Where data from different cardiac cycles is used, the data is temporally aligned.

When a full cardiac cycle or multiple cardiac cycles are available, the motion of the tracked points is not symmetrical due to the fact that the systole and diastole are generally not equal. Fourier analysis may be used to identify the initial phase (e.g., end diastole or systole) which can be used as the new timing feature. Alternatively, model-based approach may be utilized. FIG. 7 shows the cardiac cycle modeled with a piece-wise sinusoidal function. For FIG. 7, the distance as a function of time from a point on the tissue boundary to a centroid is modeled. Time shift from the beginning to a first landmark (e.g., maximum or minimum), amplitude, downtime, uptime and level are matched to the value being modeled. The downtime parameter is corresponding to systole and the uptime parameter to diastole. Additional, different or fewer model parameters may be used. Data is extracted for use in calculating spatial parameter values as a function of time over the desired time periods.

The tracked points correspond to an endocardial, epicardial or other tissue contour. For example, a plurality of points (e.g., 17 or other number) of points spaced along the endocardial boundary are tracked.

In act 32 a spatial parameter value for a point is determined as a function of time based on the tracking. In act 34, cardiac motion is characterized as a function of the spatial parameter value. The tracking, determining and/or characterizing are repeated for a plurality of points.

The tracking may alternatively correspond to segments, such as a standard cardiac left ventricle segment. The spatial parameter value is determined for the segment. The timing, motion direction, curvature and/or local ejection-fraction are determined for segments. The tracking points are grouped into segments. For instance, if using 2D ultrasound, in the apical four chamber (A4C) view, the tracked points are grouped into 6 segments (e.g., standard segments 3, 9, 14, 12, 16). A spatial parameter value associated with each segment is computed as the average, minimum, maximum, standard deviation or other combination of the spatial parameter values of the tracking points within the segment. Alternatively, the average position of the tracked points within a segment in each frame is computed. The spatial parameter values are then computed from the average position. The cardiac motion of the segment is characterized, such as by classifying the cardiac motion as a function of the spatial parameter value.

Global spatial parameter values may also or alternatively be calculated. By repeating the tracking and determining for a plurality of points, a global feature of cardiac motion may be calculated. The global feature is a function of an average, median, standard deviation, minimum, maximum or combinations thereof of the spatial parameter values for the points and/or segments included in the global calculation.

Timing is one spatial parameter value determined as a function of time. A synchronicity of cardiac motion of one or more points indicates abnormal or normal operation. The points along the left ventricle or other cardiac tissue boundary move in a consistent or synchronized manner for normal tissue.

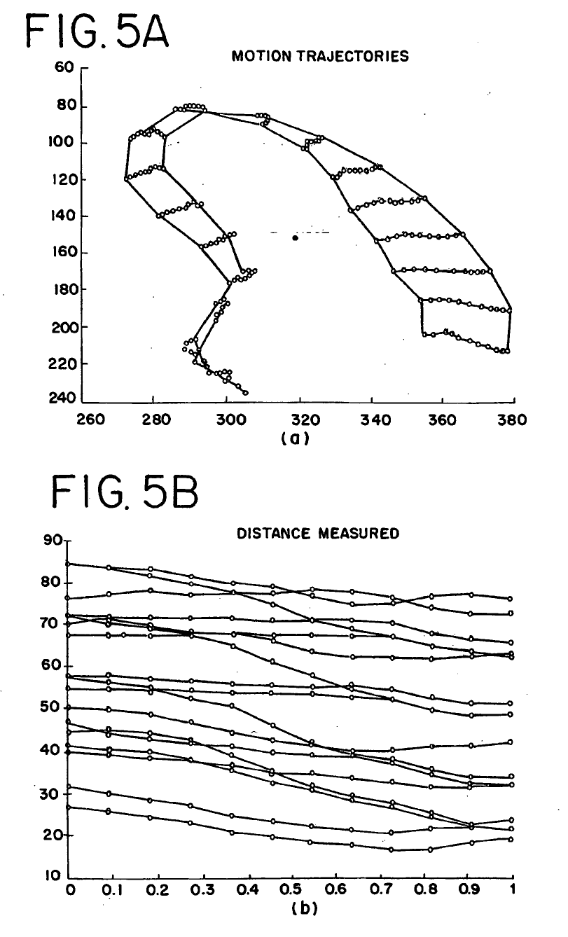

The motion trajectory for each point is provided by a distance from a reference point to the respective point as a function of time. The reference point is a centroid. The centroid varies as a function of time or a single centroid, such as associated with end diastole or systole, is selected for use throughout the sequence. FIG. 5A shows a single centroid calculated from seventeen points at end diastole. The distance of each point to the centroid is determined as a function of time. FIG. 5A shows the motion trajectories of the tracking points during systole. Where the number of frames of data available during the extracted time period is small, additional values of distance may be interpolated or identified by curve fitting.

The spatial parameter value of distance is determined as a function of time and used for identifying normal operation. For example, the time when the distance from the centroid reaches a maximum and/or minimum is identified. FIG. 5B shows the distance as a function of time for the seventeen points used in FIG. 5A. The time axis of the extracted period, such as systole, is normalized from 0 to 1. FIG. 5B shows points 9 and 10 taking more than the half of the whole systolic phase to reach their peaks, likely indicating abnormal operation. Normal operation is indicated by the distance being at a substantial maximum for end diastole and a substantial minimum for end systole.

Another indication of normal or abnormal operation is the strength of motion. The amplitude of distance of the first point to a reference point represents the strength of motion. The correlation between a cavity area and the distances may alternatively or additionally indicate normal or abnormal operation. The cavity area and distances are normalized to a same time frame. Other variation characteristics of the distance as a function of time may indicate abnormal or normal function associated with a point. While shown in FIGS. 5A and 5B for systole, variation in the distance through diastole or a whole heart cycle may be used.

The timing or synchronicity of the points relative the cardiac cycle is additionally or alternatively calculated by counting a number of the points within, outside or both within and outside a boundary of the cardiac tissue from a different time. The points which are not moving inward during the systole are identified or counted. For a given frame 1-N, an endocardial contour is determined. There are N−1 pairs of neighboring contours in time (e.g., (Ci, Ci+1), (Ci+1, Ci+2) . . . ). For normal tissue, the tracking points of Ci+1 move inward compared to the preceding Ci frame of data. The number of points of Ci+1 which are not within contour Ci may indicate abnormal operation. Similarly, the number of points within the contour of the preceding frame may indicate abnormal operation. The points within or not within indicate the location of normal or abnormal operation. The numbers are determined for each pair of sequential frames of data. The count is represented as:

$$C = \frac{{\sum }_{i + 1}^{N - 1} {out}_{i}}{( N - 1 ) M} \qquad ( 1 )$$

An average, minimum, maximum, standard deviation or other statistic of the count is determined for the sequence.

The count is a global feature. The count may also be computed by restricting the calculation to points for a segment, resulting in a local feature associated with the segment. The count is for a portion or a whole heart cycle. When diastole frames of data are available, the count is based on the points which are not moving outward.

Another spatial parameter value is the direction of motion of one or more points, such as the points shown in FIG. 5A. The direction is calculated as an average vector through the sequence. In one embodiment, Eigen values are calculated to identify movement more equal than unequal along perpendicular directions. The most significant moving direction of each point and the amount of motion in that direction is determined. The motion trajectory of a point is represented with [xi, yi], where i=0-N and N is the number of frames. The covariance matrix of xi and yi is cov(xi, yi), the two eigen values of the covariance matrix are E1 and E2 (E1=E2) and their corresponding eigen vectors are D1 and D2. D2 indicates the most significant motion direction and E2 gives the amount of motion in the direction. El shows the amount of motion in direction D1. D1 and D2 are perpendicular, but may be at other angles to each other.

Referring to FIG. 5A, if a point moves along a straight line, then E1=0, and E2 is proportional to the length of the line. A smallest ellipse which best covers all the tracked points for a given point is found. El is proportional to the short axis, and E2 is proportional to the long axis. If a point moves randomly, then E1=E2. A point with a clearly dominant motion direction is likely normal. The ratio R defined as E1/E2 identifies those points without clearly dominant motion direction as abnormal. For normal cases, R should be small.

Another spatial parameter value calculated as a function of time is the curvature associated with one or more points. A curvature through a given point is determined as a function of time. The curvature is determined from the tissue boundary. In one embodiment, the curve is determined from tissue or image data. In another embodiment, the curve is determined, at least in part, from curve fitting with adjacent points. For example, the location of adjacent points is also tracked for curve fitting through a point as a function of time.

In one embodiment, the curvature at the apex (see point 9 on FIG. 5A) is determined. In additional or alternative embodiments, the curvature at other points is determined. If a segment or tissue is dead or abnormal, it may still move because of its connection to other segments. However, the shape or curve for that point or segment may largely remain unchanged during the cardiac cycle.

In two dimensions, a plane curve v(t) is given by Cartesian parametric equations x=x(t) and y=y(t). The curvature κ is defined as:

$$κ ≡ ⅆ ϕ / ⅆ s = \frac{ⅆ ϕ / ⅆ t}{ⅆ s / ⅆ t} \qquad ( 2 )$$

where φ is the tangential angles and s is the arc length. In order to derive the dφ/dt derivative, from the identity:

$$\begin{matrix}tan ϕ = ⅆ y / ⅆ x = \frac{ⅆ y / ⅆ t}{ⅆ x / ⅆ t} = {y}^{′} / {x}^{′} & ( 3 ) \\ giving \text{:} & \\ \frac{ⅆ}{ⅆ t} ( tan ϕ ) = {sec}^{2} ϕ \frac{ⅆ ϕ}{ⅆ t} = \frac{{x}^{′} {y}^{″} - {y}^{′} {x}^{″}}{{x}^{′ 2}} . & ( 4 ) \\ Therefore , & \\ \begin{matrix}\frac{ⅆ ϕ}{ⅆ t} = \frac{1}{{sec}^{2} ϕ} \frac{ⅆ}{ⅆ t} ( tan ϕ ) \\ = \frac{1}{1 + {tan}^{2} ϕ} \frac{{x}^{′} {y}^{″} - {y}^{′} {x}^{″}}{{x}^{′ 2}} \\ = \frac{1}{1 + {y}^{′ 2} / {x}^{′ 2}} \frac{{x}^{′} {y}^{″} - {y}^{′} {x}^{″}}{{x}^{′ 2}} \\ = \frac{{x}^{′} {y}^{″} - {y}^{′} {x}^{″}}{( {x}^{′ 2} + {y}^{′ 2} )}\end{matrix} & ( 5 ) \\ Furthermore , & \\ ⅆ s / ⅆ t = \sqrt{{( \frac{ⅆ x}{ⅆ t} )}^{2} + {( \frac{ⅆ y}{ⅆ t} )}^{2} =} \sqrt{{x}^{′2} = {y}^{′2}} & ( 6 )\end{matrix}$$

Using equations 5 and 6 in Equation 2 yields:

$$κ = \frac{{x}^{′} {y}^{″} - {y}^{′} {x}^{″}}{{( {x}^{′2} + {y}^{′2} )}^{3 / 2}} \qquad ( 7 )$$

Due to the limited number of tracking points, a cubic spline interpolation of the tracking points is performed. Alternatively, the curve is determined without interpolation. The curvature at each of the tracking points in each frame is computed. In order to capture the shape change, the minimum, maximum, median, average and/or standard derivation are determined for each point of interest over the sequence of frames of data. One or more statistics of curvature characterize the curvature.

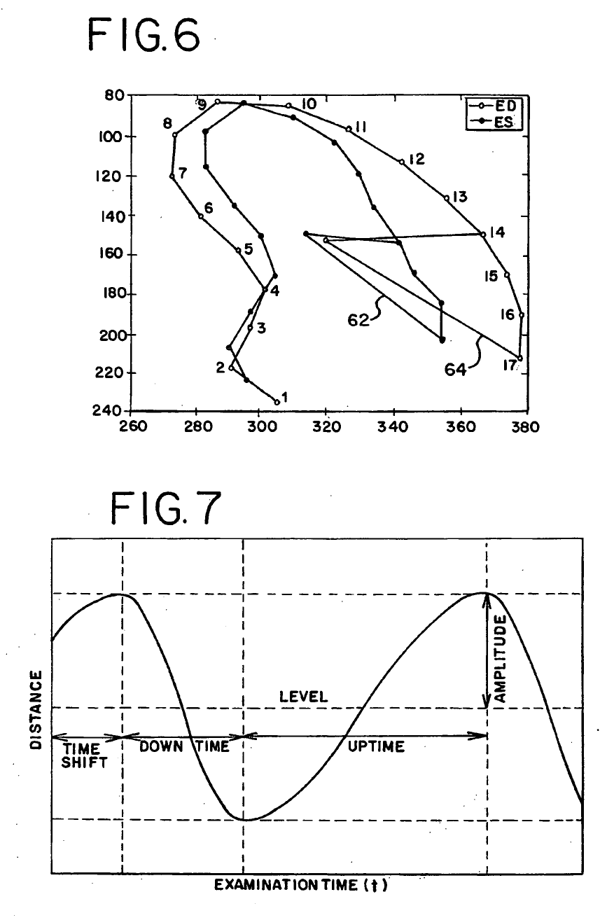

Yet another spatial parameter is the local ejection-fraction. A local area is determined. FIG. 6 shows a local area 62, 64. The local area is generally triangular shaped, but may have other shapes. For example, two points on the tissue boundary and the centroid are selected. The area bounded by the two points and the centroid or other location is calculated. The two points correspond to a segment (e.g., segment 6 as shown in FIG. 6), are adjacent, or are separated by one or more other points. To be more robust in computing local ejection-fraction ratio, one or more neighboring tracking points relative to a segment may be included. For segment 6 (points 15-17), tracking point 14 is included.

The local area is calculated at different times. In one embodiment, the different times are end diastole and end systole, but other times may be used. In FIG. 6, the local area at end diastole is labeled 62 and the local area at end systole is labeled 64. The points defining the local area are tracked. The same centroid, a subsequent centroid as shown in FIG. 6, or a different location is used. The ratio of the two local areas at different times provides the local ejection-fraction. The local ejection-fraction ratio is output. Additional local ejection fractions may be calculated. The local ejection-fraction ratio may indicate local cardiac contraction abnormalities.

Another spatial parameter is the bending energy. The contour or tissue boundary defined by the tracking points is treated as an elastic material and moving under tension. The bending energy associated with the contour may indicate the cardiac contraction strength of a segment or of the whole left ventricle.

The bending energy of the boundary is determined as a function of two or more points on the boundary. For a parametric contour v(s)=(x(t), y(t))^{T} where x and y are coordinate functions of parameter t and t is between or equal to 0 and 1. When l₁=0 and l₂=1, the bending energy of the whole contour is provided. For a segment of a contour (l₁=t=l₂), the bending energy is defined as:

$$S ( v ) = \frac{1}{2} {\int }_{{l}_{1}}^{{l}_{2}} α {\vert \frac{\partial v}{\partial t} \vert}^{2} + β {\vert \frac{{\partial }^{2} v}{\partial {t}^{2}} \vert}^{2} ⅆ t , \qquad ( 8 )$$

where α and β are two constants. The constants are weighting functions (e.g., α+β=1) selected based on user preference or application. By applying a finite element method, a discrete version of the bending energy definition is given by:

$$E ( u ) = \frac{1}{2} {u}^{T} K u \qquad ( 9 )$$

where u is the shape parameters (e.g., tracking points defining the contour) in the finite element formulation and K is the stiffness matrix.

The spatial parameter values are used alone to indicate abnormal or normal operation. Combinations of two or more spatial parameter values may be used tin indicate normal or abnormal operation. For example, the spatial parameter values are calculated and output for use by a user. As another example, an algorithm outputs an indication of normal or abnormal operation given the spatial parameter values as inputs. In one embodiment, the algorithm is a classifier or model. A second opinion or diagnosis is provided for computer assisted diagnosis based on any combination of the spatial parameter values. Clinical, other image information or other sources of data may also be used to classify the cardiac tissue operation or condition.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

I claim:

1. A method for identifying cardiac motion information from ultrasound information, the method comprising:
calculating cavity area as a function of time from ultrasound frames of data;
identifying a cardiac cycle parameter as a function of a change in the cavity area.

2. The method of claim 1 further comprising:
tracking at least a portion of a cavity border in ultrasound frames of data, where the ultrasound frames of data are associated with different portions of the cardiac cycle.

3. The method of claim 1 wherein calculating cavity area comprises:
closing a cavity border in each of the ultrasound frames of data; and
calculating the cavity area for each of the ultrasound frames of data.

4. The method of claim 1 wherein identifying the cycle parameter comprises extracting the ultrasound frames of data associated with a portion of the cardiac cycle.

5. The method of claim 4 wherein extracting comprises:
detecting inflexion points of the cavity area as a function of time; and
extracting the ultrasound frames of data associated with decreasing cavity area between inflexion points.

6. The method of claim 4 further comprising:
normalizing the extracted frames of ultrasound data as a function of time.

7. The method of claim 4 further comprising:
calculating a feature value from the extracted frames of ultrasound data; and
classifying motion as a function of the feature value.

8. A method for characterizing motion from ultrasound information, the method comprising:
tracking a first point in a sequence of ultrasound data representing at least a portion of a cycle;
determining a spatial parameter value for the first point as a function of time based on the tracking; and
characterizing motion as a function of the spatial parameter value.

9. The method of claim 8 wherein determining the spatial parameter value comprises determining a distance from a reference point to the first point as a function of time.

10. The method of claim 8 wherein tracking the first point comprises tracking the first point through a sequence including at least systole portions of a cardiac cycle, the first point associated with an endocardial contour.

11. The method of claim 9 wherein determining the distance comprises determining the distance from the first point to a centroid.

12. The method of claim 9 further comprising:
repeating the tracking, determining and characterizing for a plurality of points including the first point.

13. The method of claim 8 wherein characterizing motion comprises determining a synchronicity of variation of the distance as a function of time with a cardiac cycle.

14. The method of claim 8 wherein determining the spatial parameter value comprises determining amplitudes of distance of the first point to a reference point, a correlation between an area and the distances or combinations thereof.

15. The method of claim 12 wherein determining the spatial parameter value comprises counting a number of the plurality of points within, outside or both within and outside a boundary of the tissue from a different time.

16. The method of claim 8 wherein determining the spatial parameter value comprises determining a direction of movement of the first point.

17. The method of claim 16 wherein determining the direction comprises calculating first and second eigen values.

18. The method of claim 16 wherein characterizing comprises identifying movement more equal than unequal along perpendicular directions.

19. The method of claim 8 wherein characterizing comprises classifying the cardiac motion as a function of the spatial parameter value.

20. The method of claim 8 wherein determining the spatial parameter comprises calculating a curvature through the first point as a function of time.

21. The method of claim 20 further comprising:
tracking second and third points associated with cardiac tissue in the sequence of ultrasound data;
wherein calculating the curvature comprises fitting a curve to the first, second and third points.

22. The method of claim 20 wherein characterizing the motion comprises characterizing as a function of a minimum, a maximum, a median, an average, a standard deviation or combinations thereof of the curvature.

23. The method of claim 8 wherein tracking comprises tracking the first point, a second point and additional points on a boundary of cardiac tissue, wherein determining the spatial parameter value comprises determining first and second local areas as a function of the first point and the second point on the boundary at different times.

24. The method of claim 23 wherein characterizing comprises outputting a local ejection-fraction ratio as a function of the first and second local areas.

25. The method of claim 23 wherein the different times are end diastole and end systole.

26. The method of claim 8 wherein tracking comprises tracking the first point, a second point and additional points on a boundary of cardiac tissue, wherein determining the spatial parameter value comprises determining bending energy of the boundary as a function of the first point and the second point on the boundary.

27. The method of claim 8 wherein tracking the first point comprises tracking a segment of cardiac tissue, wherein determining the spatial parameter value for the first point comprises determining the spatial parameter value of the segment, and wherein characterizing the motion comprises characterizing cardiac motion of the segment.

28. The method of claim 8 further comprising:
repeating the tracking and determining for a plurality of points;
calculating a global feature as a function of the spatial parameter values for the plurality of points, the global feature being a function of an average, median, standard deviation, minimum, maximum or combinations thereof of the spatial parameter values.

29. The method of claim 10 wherein the sequence includes a full cardiac cycle.

30. The method of claim 29 wherein the sequence includes a plurality of cardiac cycles;
further comprising:
temporally aligning the ultrasound data for different ones of the plurality of cardiac cycles.

31. In a computer readable storage media having stored therein data representing instructions executable by a programmed processor for characterizing cardiac motion from ultrasound information, the storage media comprising instructions for:
tracking a first point associated with cardiac tissue in a sequence of ultrasound data representing at least a portion of a heart;
determining a spatial parameter value for the first point as a function of time based on the tracking; and
characterizing cardiac motion as a function of the spatial parameter value.

32. The instructions of claim 31 wherein determining the spatial parameter value comprises determining a distance from a centroid to the first point as a function of time, and wherein characterizing cardiac motion comprises determining a synchronicity of variation of the distance as a function of time with a cardiac cycle.

33. The instructions of claim 31 further comprising:
repeating the tracking, determining and characterizing for a plurality of points including the first point;
wherein determining the spatial parameter value comprises counting a number of the plurality of points within, outside or both within and outside a boundary of the cardiac tissue from a different time.

34. The instructions of claim 31 wherein determining the spatial parameter value comprises calculating first and second eigen values representing a direction of movement of the first point, and wherein characterizing comprises identifying movement more equal than unequal along perpendicular directions.

35. The instructions of claim 31 wherein characterizing comprises classifying the cardiac motion as a function of the spatial parameter value.

36. The instructions of claim 31 wherein determining the spatial parameter comprises calculating a curvature through the first point as a function of time, and wherein characterizing the cardiac motion comprises characterizing as a function of a minimum, a maximum, a median, an average, a standard deviation or combinations thereof of the curvature.

37. The instructions of claim 31 wherein tracking comprises tracking the first point, a second point and additional points on a boundary of the cardiac tissue, wherein determining the spatial parameter value comprises determining first and second local areas as a function of the first point and the second point on the boundary at end diastole and end systole, and wherein characterizing comprises outputting a local ejection-fraction ratio as a function of the first and second local areas.

38. The instructions of claim 31 wherein tracking comprises tracking the first point, a second point and additional points on a boundary of the cardiac tissue, wherein determining the spatial parameter value comprises determining bending energy of the boundary as a function of the first point and the second point on the boundary.

39. A method for characterizing motion from ultrasound information, the method comprising:
tracking a first point associated with cardiac tissue in a sequence of ultrasound data representing at least a portion of a heart;
determining two or more different types of parameter values for the first point as a function of time based on the tracking; and
characterizing cardiac motion as a function of the two or more different types of parameter values.
